(19) United States
(12) Patent Application Publication
(10) Pub. No.: US 2012/0273586 A1
(43) Pub. Date: Nov. 1, 2012

(54) Plasticized Elastomer Including a Volatile Compound

(76) Inventors: Mark R. Shook, Phoenix, AZ (US); Mark Grodsky, Phoenix, AZ (US)

(21) Appl. No.: 13/457,375

(22) Filed: Apr. 26, 2012

Related U.S. Application Data
(60) Provisional application No. 61/481,134, filed on Apr. 29, 2011.

(51) Int. Cl. B05B 1/24; A01P 1/00; A61K 47/32; A01N 25/10; A61L 9/01

(52) U.S. Cl. 239/13; 512/4; 514/772.6; 206/223; 206/570

(57) ABSTRACT

A plasticized elastomer includes a polymeric carrier and a volatile compound, such as a fragrance, medicament, or antimicrobial, disposed in the polymeric carrier. The polymeric carrier absorbs microwave energy and slowly dissipates it as heat until the polymeric carrier reaches thermal equilibrium with the ambient temperature. The heat of the polymeric carrier increases a vapor pressure of the volatile compound, which boosting its evaporation into the ambient environment.

### CROSS-REFERENCES TO RELATED APPLICATIONS

This application claims priority to, and the benefit of, U.S. Patent Application Ser. No. 61/481,134, entitled “Plasticized Elastomer Comprising a Polymeric Carrier in Combination with a Volatile Substance, and Receptacle, and Method Using Same,” filed Apr. 29, 2011, the entire contents of which are incorporated herein by reference.

### FIELD

Embodiments generally relate to a plasticized elastomer and methods, systems, devices, and compositions regarding same, and more particularly, to a polymer carrier in combination with a volatile compound; and most particularly to a noncrystalline polymer that acts both as a thermal battery and a carrier of a volatile compound such as a fragrance, a medicament, or an antimicrobial.

### BACKGROUND

The utility of some substances may rely, in part, on their volatility characteristics. For example, some medicaments are administered through the olfactory system of a patient and, therefore, are effective when vaporized. Similarly, the sphere of influence for an antimicrobial spray or vapor depends on its spatial range of release. In yet another example, the value of a fragrance for domestic use may depend on its rate and range of release into an ambient environment. In each example, prolonged release may be desirable. For example, the medicament may need to be administered over hours or days, the antimicrobial compound may also need to be administered over hours or days, and users of the domestic fragrance may favor prolonged fragrance dissipation.

Accordingly, it would be an advance in the art to provide methods, devices, compounds, or systems that prolong the release of a substance into an ambient environment.

### SUMMARY

In certain embodiments, a plasticized elastomer for dispensing a volatile compound includes a volatile compound disposed in the polymeric carrier having a specific heat capacity above 1.0 J/(g·° K).

In certain embodiments, a product for dispensing a volatile compound includes a volatile compound disposed in the polymeric carrier configured to be housed in a receptacle. The polymeric carrier is configured to slowly dissipate imposed heat. The volatile compound is configured to evaporate while the polymeric carrier dissipates heat.

In certain embodiments, a method for dispensing a volatile compound includes exposing a plasticized elastomer to microwave energy to increase a temperature of the plasticized elastomer. The plasticized elastomer includes a polymeric carrier and a volatile compound disposed in the polymeric carrier. The method further includes allowing the plasticized elastomer to dissipate heat until a temperature of the plasticized elastomer reaches thermal equilibrium with an ambient temperature.

In certain embodiments, a device elevates the vapor pressure of a volatile compound. The device includes a polymeric carrier disposed within a receptacle. The polymeric carrier is impregnated with a volatile compound. The polymeric carrier absorbs microwave energy and dissipates it as heat, elevating its temperature and, in turn, increasing the vapor pressure of the impregnated volatile compound above its vapor pressure at ambient temperature.

### BRIEF DESCRIPTION OF THE DRAWINGS

The invention will be better understood from a reading of the following detailed description taken in conjunction with the drawings in which like reference designators are used to designate like elements, and in which:

FIG. 1 is a graph illustrating vapor pressure profiles for a plurality of substances;

FIG. 2 is a graph illustrating the crystallinity of Ethylene Vinyl Acetate (EVA) and Ethylene Acrylate graphed against comonomer percent weight;

FIG. 3 is a schematic illustrating a system 300 for facilitating release of a volatile compound;

FIG. 4 is a flow chart of a method for facilitating release of a volatile compound using the system of FIG. 3;

FIGS. 5a-5d are schematics of exemplary receptacles described in FIG. 3;

FIGS. 6a-10b are graphs illustrating thermal properties of a receptacle and polymeric carrier; and

FIG. 11 is a graph illustrating melting points in degrees Fahrenheit versus weight percent vinyl acetate for Applicants' EVA elastomer.

### DETAILED DESCRIPTION OF THE PREFERRED EMBODIMENTS

Embodiments are described in the following description with reference to the FIGs. in which like numbers represent the same or similar elements. Reference throughout this specification to “one embodiment,” “an embodiment,” or similar language means that a particular feature, structure, or characteristic described in connection with the embodiment is included in at least one embodiment of the present invention. Thus, appearances of the phrases “in one embodiment,” “in an embodiment,” and similar language throughout this specification may, but do not necessarily, all refer to the same embodiment.

The described features, structures, or characteristics of the invention may be combined in any suitable manner in one or more embodiments. In the following description, numerous specific details are recited to provide a thorough understanding of embodiments of the invention. One skilled in the relevant art will recognize, however, that the invention may be practiced without one or more of the specific details, or with other methods, components, materials, and so forth. In other instances, well-known structures, materials, or operations are not shown or described in detail to avoid obscuring aspects of the invention.

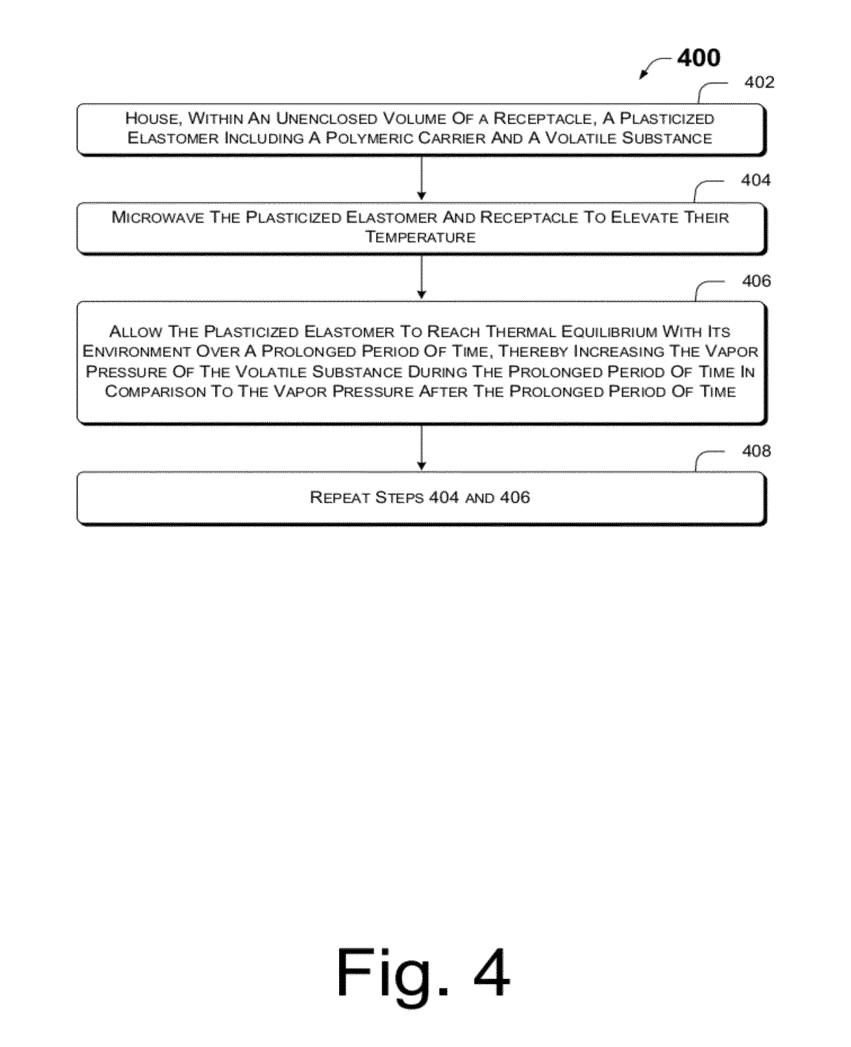

The schematic flow chart diagrams included are generally set forth as a logical flow-chart diagram (e.g., FIG. 4). As such, the depicted order and labeled steps are indicative of one embodiment of the presented method. Other steps and methods may be conceived that are equivalent in function, logic, or effect to one or more steps, or portions thereof, of the illustrated method. Additionally, the format and symbols employed are provided to explain the logical steps of the method and are understood not to limit the scope of the method. Although various arrow types and line types may be employed in the flow-chart diagrams, they are understood not to limit the scope of the corresponding method (e.g., FIG. 4). Indeed, some arrows or other connectors may be used to indicate only the logical flow of the method. For instance, an arrow may indicate a waiting or monitoring period of unspecified duration between enumerated steps of the depicted method. Additionally, the order in which a particular method occurs may or may not strictly adhere to the order of the corresponding steps shown.

The volatility of a substance depends on its vapor pressure characteristics. At a given temperature, a substance with a lower vapor pressure vaporizes (e.g., via vaporization of a liquid to a gas or sublimation of a solid to a gas) less readily than a substance with a higher vapor pressure.

Liquids may change to a vapor at temperatures below their boiling points through the process of evaporation. Evaporation is a surface phenomenon in which molecules located near the liquid's edge, not contained by enough liquid pressure on that side, escape into the surroundings as vapor.

On the other hand, boiling is a process in which molecules anywhere in the liquid escape, resulting in the formation of vapor bubbles within the liquid. The boiling point of an element or a substance is the temperature at which the vapor pressure of the liquid equals the environmental pressure surrounding the liquid. A liquid in a vacuum environment has a lower boiling point than when the liquid is at atmospheric pressure. A liquid in a high pressure environment has a higher boiling point than when the liquid is at atmospheric pressure. The boiling point of a liquid varies dependent upon the surrounding environmental pressure. Different liquids (at a given pressure) boil at different temperatures.

The normal boiling point of a liquid is the special case in which the vapor pressure of the liquid equals the defined atmospheric pressure at sea level, 1 atmosphere. At that temperature, the vapor pressure of the liquid becomes sufficient to overcome atmospheric pressure and lift the liquid to form bubbles inside the bulk of the liquid. The standard boiling point is now (as of 1982) defined by the International Union of Pure and Applied Chemistry (IUPAC) as the temperature at which boiling occurs under a pressure of 1 bar.

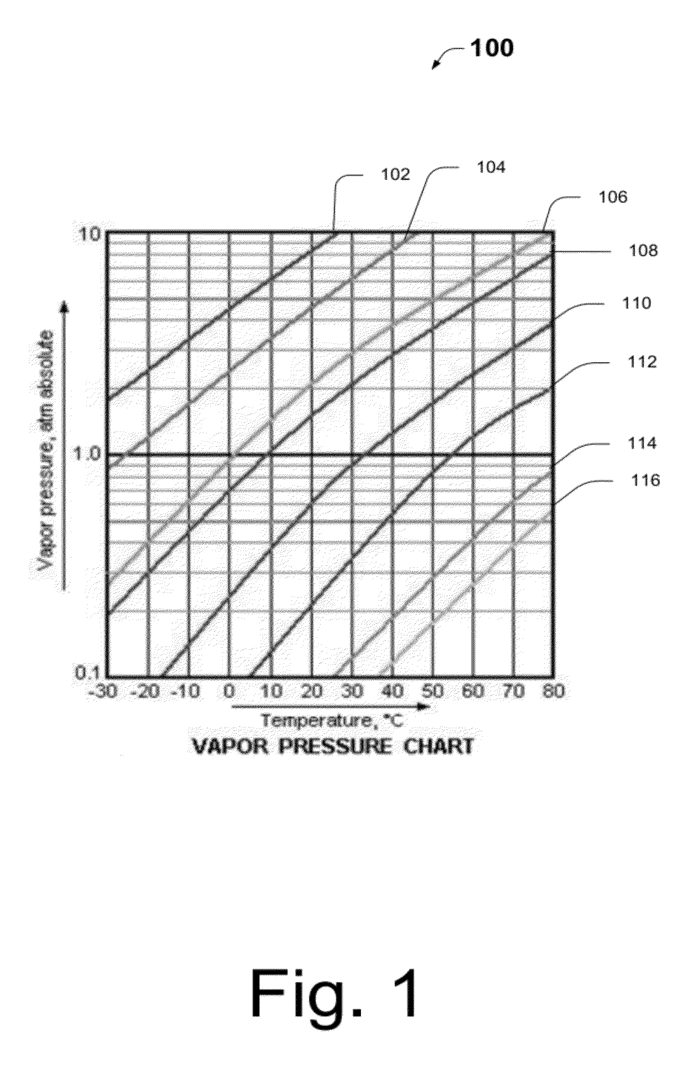

The heat of vaporization is the amount of energy required to convert or vaporize a saturated liquid (i.e., a liquid at its boiling point) into a vapor. Referring to FIG. 1, a graph 100 illustrates vapor pressure profiles for a plurality of substances: Propane 102, Methyl Chloride 104, Butane 106, Neo-Pentane 108, Diethyl Ether 110, Methyl Acetate 112, Flurobenzene 114, and 2-Heptene 116. For each substance of FIG. 1, the vapor pressure increases with an increase in temperature. Consequently, the substances vaporize more readily as the temperature increases. In certain embodiments, a polymer is used as a carrier (“polymeric carrier”) for a volatile compound. By “volatile compound,” Applicants mean a compound comprising a boiling point of about 80° C. or less at one atmosphere. For example, the polymeric carrier may be impregnated or infused with a fragrance, an antimicrobial, or a medicament. The polymeric carrier is heated for a period of time, such as through the use of a microwave oven for a period between a few seconds to five minutes, for example. Thereafter, the elevated temperature of the polymeric carrier promotes release of the volatile compound into the ambient environment over a prolonged period of time as the polymeric carrier gradually reaches thermal equilibrium with its ambient environment (e.g., ambient temperature).

In one example, the polymeric carrier is a copolymer of ethylene and vinyl acetate, such as Ethylene Vinyl Acetate (“EVA”). EVA is an odorless copolymer with a specific density of about 0.922-0.945 g/cm³. In some embodiments, the weight percent of vinyl acetate in EVA varies from 10-40% with the remainder being ethylene.

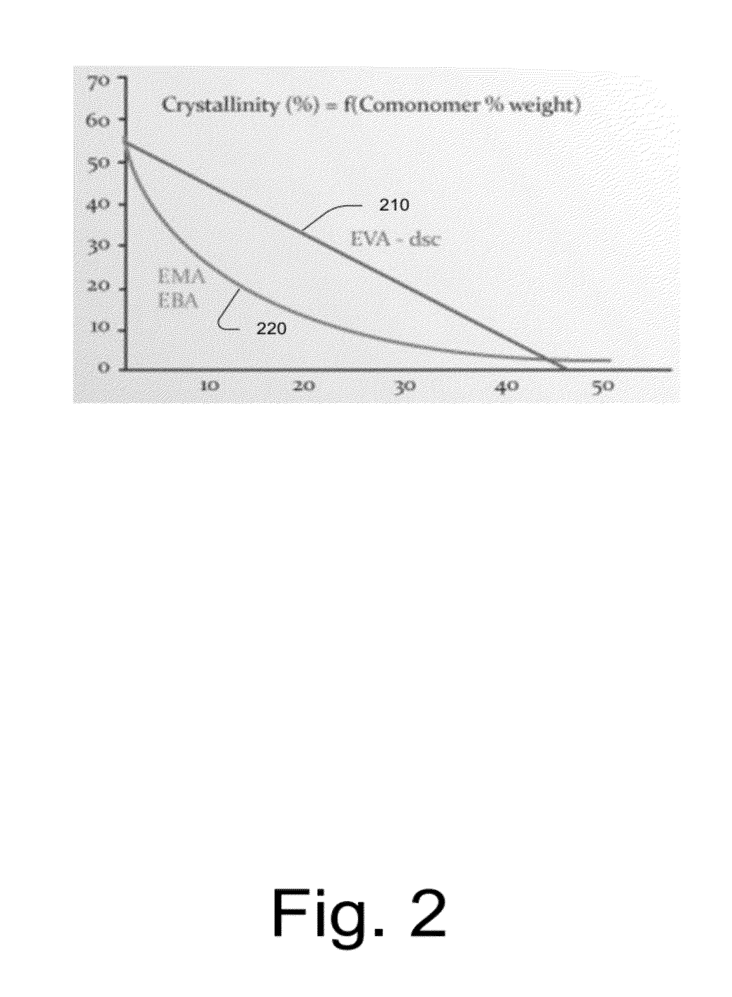

The copolymer content also impacts polymer cystallinity. Referring to FIG. 2, the crystallinity of EVA 210 and ethylene acrylate 220 is graphed against comonomer % weight. The EVA 210 has elastomeric properties yet can be processed like other thermoplastics.

The chemical structure of EVA I includes an ester moiety that is pendant to a carbon chain.

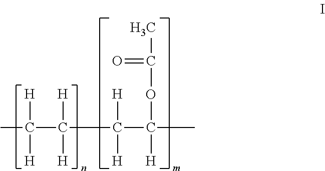

The longer the polymer chain the higher its viscosity, and hence the lower its melt flow index, resulting in greater mechanical strength and a higher ring and ball softening point.

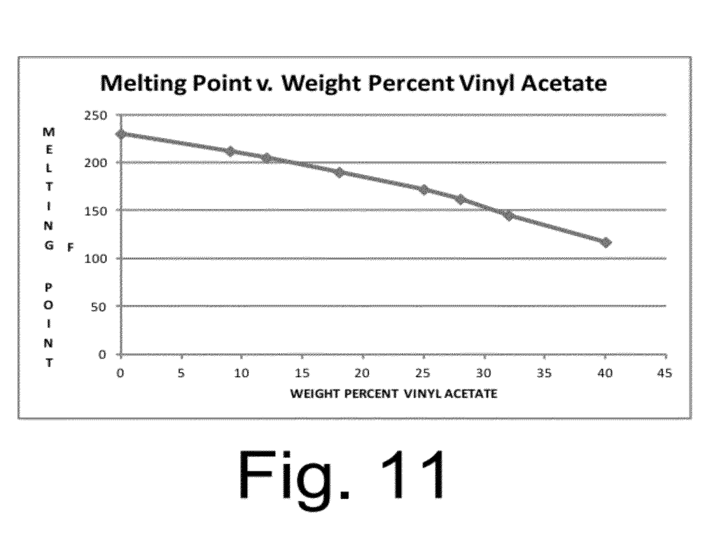

FIG. 11 graphically shows the melting point in degrees Fahrenheit versus weight percent vinyl acetate for Applicants' EVA elastomer. In certain embodiments, Applicants' composition comprises an EVA copolymer comprising about 10 weight percent vinyl acetate groups. That EVA copolymer comprises a melting point of about 212° F. In certain embodiments, Applicants' composition comprises an EVA copolymer comprising about 25 weight percent vinyl acetate groups. That EVA copolymer comprises a melting point of about 172° F. In certain embodiments, Applicants' composition comprises an EVA copolymer comprising about 40 weight percent vinyl acetate groups. That EVA copolymer comprises a melting point of about 117° F.

Those skilled in the art will appreciate in any of these EVA embodiments, the degree of crystallinity is low. As a result, all of these EVA copolymers comprise glasses rather than crystalline solids. This being the case, each of these EVA embodiments may be heated to about 400° F. and, even though about the glass transition temperature Tg, and even though above the respective melting point, these copolymers remain stable and do not liquify.

In certain embodiments, Applicants' polymeric carrier comprises polyvinylacetate, wherein n=0 in structure I hereinabove. Polyvinylacetate has a Tg of about 38-40° C. In certain of these embodiments, a fragrance, medicament, antimicrobial compound, or any combination thereof, is disposed in the polyvinylacetate.

In certain embodiments, Applicants' polymeric carrier comprises polyvinylbutyrate. Polyvinylbutyrate comprises a lower glass transition temperature than does polyvinylacetate. In certain embodiments, Applicants dissolve ethyl butyrate in polyvinylbutyrate. Ethyl butyrate is one of the most common chemicals used in flavors and fragrances.

When exposed to microwave energy, the pendent ester moieties in Applicants' polymeric carrier spin at a high frequency, converting the microwave energy to heat without melting, even at high temperatures. Rather than melting, EVA undergoes a glass transition from a substantially solid material to a rubber-like state. For example, depending on the weight percent of the vinyl acetate, EVA can be heated to temperatures of 100-400° F. without melting.

Given that EVA does not melt at high temperatures, EVA has little to no vapor pressure at temperatures such as 400° F. and below in comparison to volatile compounds at such temperatures. EVA can be a good carrier of volatile compounds because, while the volatile compound substantially vaporizes at elevated temperatures, EVA substantially does not, in certain embodiments.

In certain embodiments, the polymeric carrier has the effect of a thermal battery.

The polymeric carrier has a high heat capacity, which measures a quantity of heat needed to change a substance's temperature. Specific heat capacity is the heat capacity per unit mass of the substance. Substances with a low heat capacity dissipate energy quickly for a given pressure while substances with a high heat capacity dissipate heat slowly for the given pressure. Here, the polymeric carrier has a (mass) specific heat capacity of above 1000 J/(Kg·° K). To illustrate, EVA has a high heat capacity, an average specific heat capacity of about 2220 J/(Kg ° K). When the temperature of EVA is elevated, its heat dissipates over a prolonged period of time in proportion to its mass. Therefore, Applicants have found that their polyvinvyl ester polymeric carriers can act as a thermal battery due to its high heat capacity.

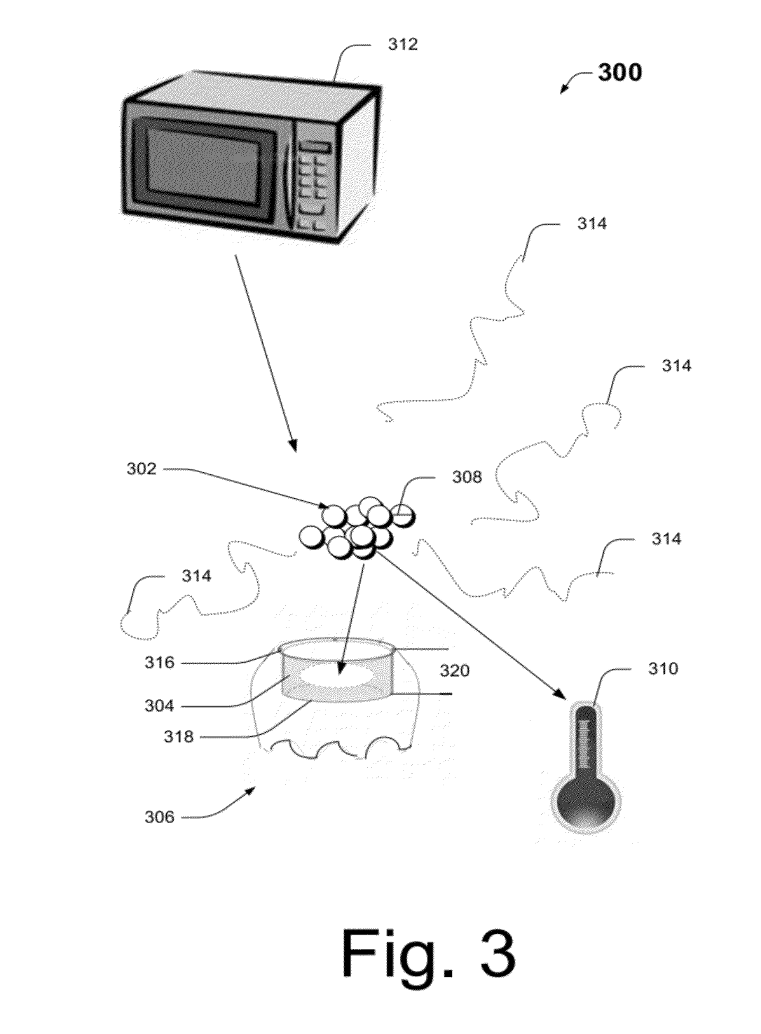

In certain embodiments, Applicants dispose a volatile compound into their polymeric carrier to form a plasticized elastomer. Referring to FIGS. 3 and 4, in certain embodiments, the plasticized elastomer 302 is housed and microwave heated in a receptacle 306, elevating the temperature of the polymeric carrier and, in turn, the vapor pressure of the volatile compound 314. At a step 402 of method 400 in FIG. 4, the plasticized elastomer 302 is disposed within an unenclosed portion 304 of a receptacle 306. The plasticized elastomer 302 is illustrated in FIG. 3 as pellets of about three millimeters diameter 308 each. The pellets may be of any suitable shape. Alternatively, or in combination, the plasticized elastomer 302 is in a form of a paste, gel, a spray, a solid material, or a combination thereof. In certain embodiments, a ratio of about one gram plasticized elastomer 302 is disposed in about five milliliters unenclosed portion 304 to about one gram plasticized elastomer 302 to about 12 milliliters unenclosed portion 304, for example. In certain embodiments, about four grams of plasticized elastomer 302, with about a 20% fragrance load, are disposed in the unenclosed portion 304 having about a one and a half inch diameter base 318, about a two and a quarter inch diameter top 316, and a one and a quarter inch height 320.

At a step 404 of method 400, the plasticized elastomer 302 and the receptacle 306 are heated in a microwave oven 312. In certain embodiments, plasticized elastomer 302 and the receptacle 306 are heated in the microwave oven 312 for a period of time between a few seconds (e.g., 2 seconds) to about ten minutes. In certain embodiments, plasticized elastomer 302 and the receptacle 306 are heated in a microwave oven 312 for a period of time between about thirty seconds to eight minutes. In certain embodiments, plasticized elastomer 302 and the receptacle 306 are heated in a microwave oven 312 for a period of time between about forty five seconds to four minutes. In certain embodiments, plasticized elastomer 302 and the receptacle 306 are heated in a microwave oven 312 for a period of time between about one minute to three minutes. In certain embodiments, plasticized elastomer 302 and the receptacle 306 are heated in a microwave oven 312 for a period of time between about one to three minutes.

A standard domestic microwave oven 312 heats its content by passing non-ionizing microwave radiation at a frequency of about 2.45 GHz. The content absorbs the energy from the microwaves by dielectric heating. During dielectric heating, molecules that have electric dipoles rotate as they align themselves with the alternating electric field of the microwave. The rotation produces heat. Domestic microwaves operate at about 600 to about 1,400 watts, which can heat a retentive substance to about 175° F. to about 400° F. when used between about 2 to about 6 minutes.

Applicants' plasticized elastomer 302 absorbs the microwave energy and converts that energy to heat, such that, after the period of time described above, the surface temperature 310 of the plasticized elastomer 302 is between about 100° F. and about 400° F. In certain embodiments, the surface temperature 310 of the plasticized elastomer 302 is between about 200° F. and about 350° F. In certain embodiments, the surface temperature 310 of the plasticized elastomer 302 is between about 250° F. and about 325° F. The elevated temperature of the polymeric carrier increases the temperature of the impregnated volatile compound and, in turn, the vapor pressure of the impregnated volatile compound.

At a step 406, the plasticized elastomer 302 is allowed to cool over a prolonged period of time, reaching thermal equilibrium with its environment. In certain embodiments, the plasticized elastomer 302 reaches thermal equilibrium with its environment between ten minutes to five hours. In certain embodiments, the plasticized elastomer 302 reaches thermal equilibrium with its environment between thirty minutes to two hours. The volatile compound continues to evaporate/vaporize over the entire cool-down period. At step 408, the steps 404 to 406 are repeated in order to further promote vaporization of the volatile compound.

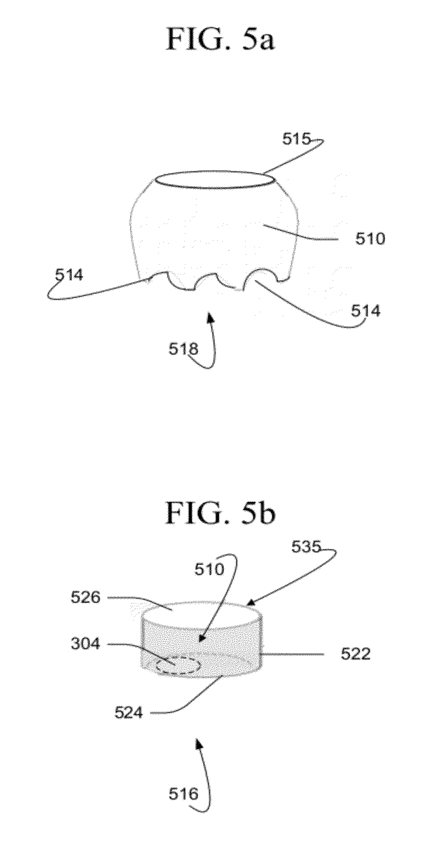

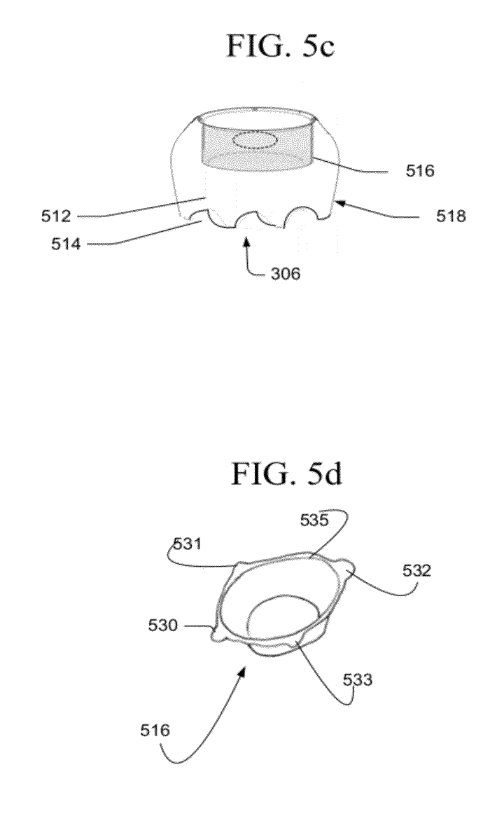

Referring to FIGS. 5a-5d, Applicants' receptacle 306 is illustrated in further detail. Receptacle 306 comprises an inner vessel 516 having an open top 304 disposed within an outer vessel 518.

Referring to FIG. 5a, outer vessel 518 is formed to include a concavity and/or a periphery 515 which defines an open top. In certain embodiments, outer vessel 518 comprises a contiguous bottom defining an enclosed space (not shown). The enclosed space includes air or other insulating material (e.g., polyurethane or polystyrene foam). In certain embodiments, the enclosed space is under vacuum.

In other embodiments and as illustrated in FIG. 5a, outer vessel 518 does not include a contiguous bottom. In certain embodiments, outer vessel 518 is formed to include vents 514 on a surface of the receptacle to increase air flow within space 512.

Referring to FIG. 5b, inner vessel 516 comprises a cylindrical wall 522, a bottom 524, and a periphery 535 that defines an open top 526. In the illustrated embodiment of FIG. 5b, inner vessel 516 comprises a cylindrical shape. In other embodiments, inner vessel 516 comprises an alternate configuration selected from the group consisting of a parallelepiped, a sphere, a prism, and/or a combination thereof. The inner vessel 516 defines a space 510.

Referring now to FIG. 5c, in certain embodiments inner vessel 516 is disposed within outer vessel 518 wherein periphery 535 of inner vessel 518 is sealingly attached to periphery 515 of outer vessel 518. In other embodiments, outer vessel 518 is formed such that a top portion 526 of inner vessel 516 and a top portion 520 of outer vessel 518 are not in contiguous physical attachment or connection. For example in the illustrated embodiment of FIG. 5d, periphery 535 of inner vessel 516 is formed to include tabs 530, 531, 532, and 533, extending outwardly therefrom. In certain embodiments, tabs 530, 531, 532, and 533, rest on periphery 515 of outer vessel 518. In certain embodiments, tabs 530, 531, 532, and 533, fit and/or snap into corresponding apertures formed in the outer vessel 518.

In other embodiments, inner vessel 516 comprises fewer than 4 tabs extending outwardly from periphery 535. In still other embodiments, inner vessel 516 comprises more than 4 tabs extending outwardly from periphery 535.

In some embodiments, the receptacle 306 may be molded as an integral assembly. Alternatively, inner vessel 516 is formed from a first material and the outer vessel 518 is formed from a second, and different, material.

The receptacle 306 may be formed of any suitable material that has heat retentive properties and remains substantially a solid when heated, such as wood, metal, plastic, or composite materials. Preferably, the heat retentive receptacle 306 is microwave safe such that it does not melt or cause a fire when heated in a microwave. In certain embodiments, the material of the inner vessel 516 and the material of the outer vessel 518 have substantially similar physical properties. In other embodiments, the material of the inner vessel 516 and the material of the outer vessel 518 have different physical properties.

In one embodiment, the inner vessel 516 may be made of ceramic while the outer vessel 518 is made of polypropylene. Preferably, the outer vessel 518 allows microwave energy to pass through the material without the material of the outer vessel 518 increasing in temperature significantly, such as causing less than 20 degrees Fahrenheit temperature rise of a material of the outer vessel 518. In this manner, when the receptacle 306 is removed from the microwave, it does not burn its user. Typical materials that transfer microwave energy without substantially increasing in temperature include: polyolefin, polyproplyene, polyethylene, poly carbonate, and acrylonitrile butadiene styrene copolymer.

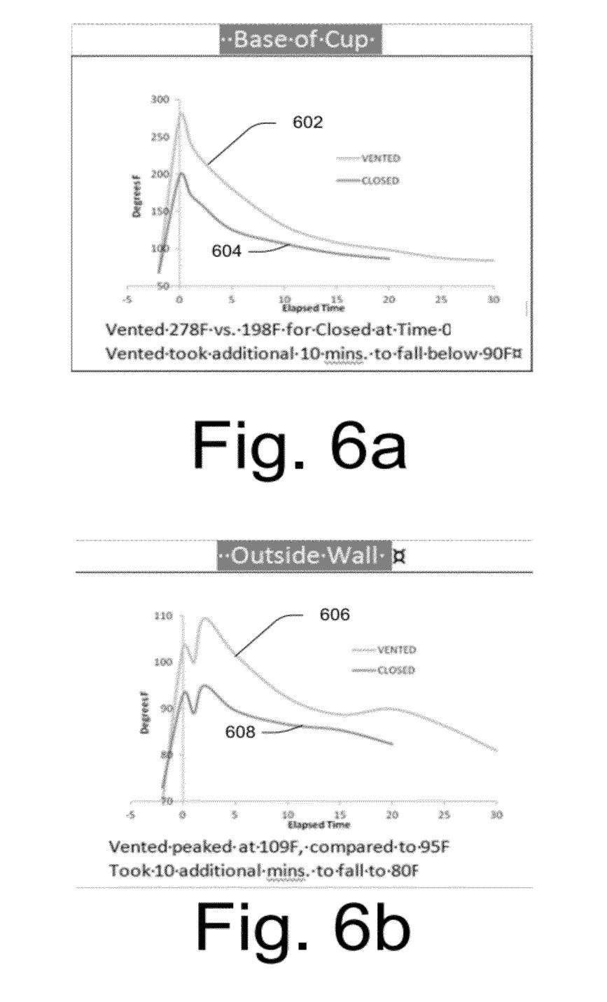

FIGS. 6-10 graphically recite thermal properties of receptacle 306 and a plasticized elastomer, including Ethylene Vinyl Acetate, under various conditions. Temperature readings were taken before heating (Time=−2 mins.), immediately after heating (time=0) and at prescribed intervals thereafter.

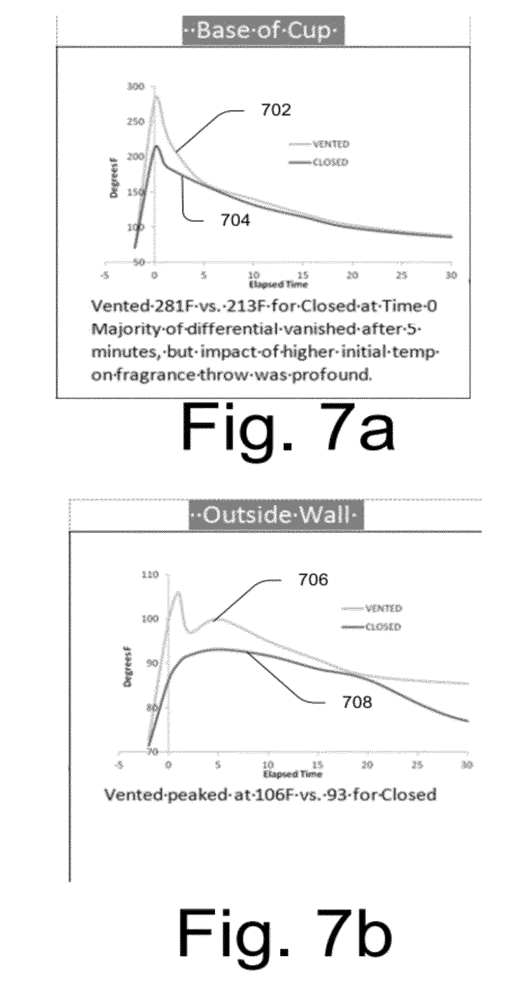

Referring to FIGS. 5a-5d and 6a-6b, the thermal properties of an empty receptacle 306 having an open bottom are illustrated for two conditions: with and without vents 514. Here, the receptacle 306 is placed in a 1200-watt microwave oven and heated at full power for 2 minutes. FIG. 6a shows the heat dissipation measured at a base 524 of the inner vessel 516 (“cup”), while FIG. 6b shows the heat dissipation measured at the outer wall 528 of the vessel 518. Lines 602 and 606, show the thermal properties of the receptacle 306 with vents 514, while Lines 604 and 608 show the thermal properties of the receptacle without vents 514. Here, the vented receptacle 306 took an additional ten minutes to fall below 90° F. than did the unvented receptacle 306. Consequently, the vents 514 promoted a prolonged elevated temperature. Referring to FIGS. 5a-5d and 7a-7b, the thermal properties of the receptacle 306 having an open bottom and loaded with the plasticized elastomer 302 are illustrated for two conditions: with and without vents 514. Here, 2.9 grams of 25%-fragranced EVA pellets were loaded into the receptacle 306, placed in a 1200-watt microwave oven, and heated at full power for 2 minutes. FIG. 7a shows the heat dissipation measured at the base 524 of the inner vessel 516 (“cup”), while FIG. 7b shows the heat dissipation measured at the outer wall 528 of the outer vessel 518. Lines 702 and 706, show the thermal properties of the receptacle 306 with vents 514, while Lines 704 and 708 show the thermal properties of the receptacle without vents 514.

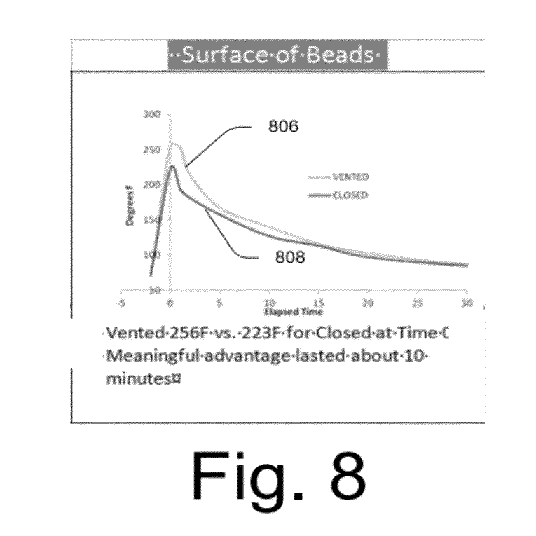

FIG. 8 shows heat dissipation measured at the surface of the plasticized elastomer 302. Here, readings for the vented receptacle 306 loaded with plasticized elastomer 302 exceeded those of the unvented (“closed”) receptacle 306 loaded with plasticized elastomer 302. Here, the receptacle 306 with vents 514 had a measurable advantage in heat retention in comparison to the unvented receptacle 306.

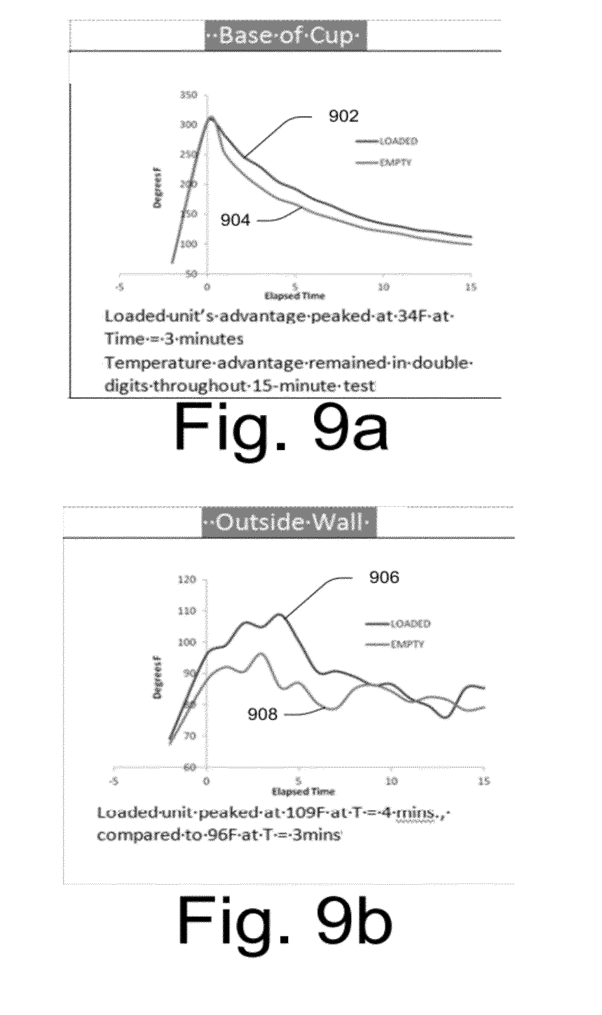

Referring to FIGS. 5a-5d and 9a-9b, the thermal properties of the receptacle 306 having an open bottom and vents 514 are illustrated for two conditions: housing and not housing the plasticized elastomer 302. FIG. 9a shows the heat dissipation measured at the base 524 of the inner vessel 516 (“cup”), while FIG. 9b shows the heat dissipation measured at the outer wall 528 of the outer vessel 518. Lines 902 and 906, show the thermal properties of the receptacle 306 housing the plasticized elastomer 302, while Lines 904 and 908 show the thermal properties of the receptacle 306 without housing the plasticized elastomer 302. Initial cup base temperatures were comparable. Here, the loaded, vented receptacle 306 retained heat better than did the empty, vented receptacle 306.

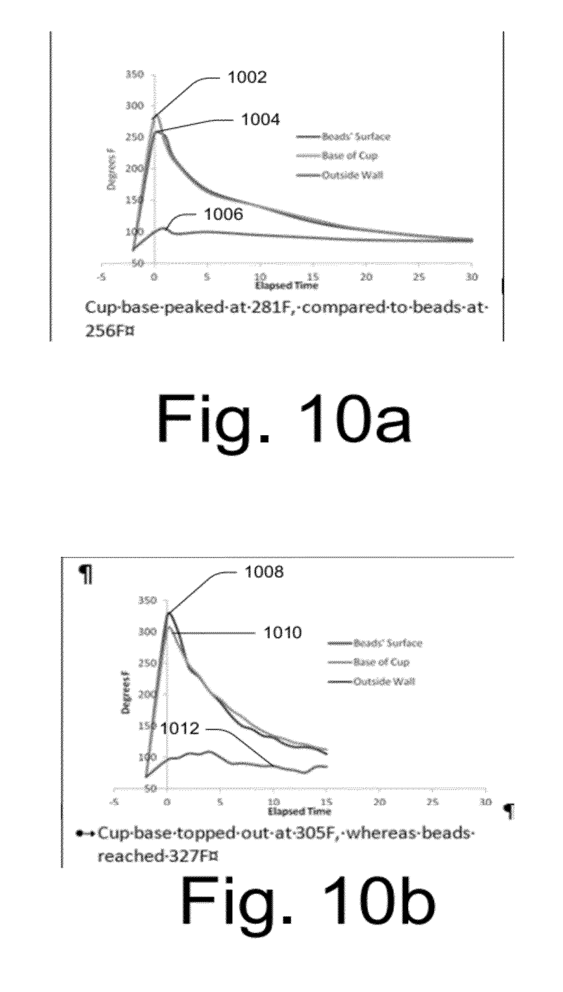

Referring to FIGS. 5a-5d and 10a-10b, the thermal properties of the receptacle 306 having an open bottom, vents 514, and loaded with plasticized elastomer 302 are illustrated for two different dates: FIG. 10a shows the heat dissipation measured on Jan. 2, 2011, while FIG. 10b show heat dissipation measured on Jan. 16, 2011. Lines 1002 and 1008 show the thermal properties of the plasticized elastomer 302 over time; lines 1004 and 1010 show the thermal properties of the base 524 of the inner vessel 516 (“cup”); and lines 1006 and 1012 show the thermal properties of the outer vessel 518 measured at the outer wall 528.

TABLES 1, 2, and 3 below recite the data graphically shown in FIGS. 6-10.

TABLE 1
(1/2/11 Data)

| | VENTED-Loaded | | | CLOSED-Loaded | | |
|---|---|---|---|---|---|---|
| Time | Base of | Beads′ | Outside | Base of | Beads′ | Outside |
| (mins.) | Cup | Surface | Wall | Cup | Surface | Wall |
| −2 | 71 | 70 | 73 | 71 | 71 | 71 |
| 0 | 281 | 256 | 100 | 213 | 223 | 86 |
| 1 | 241 | 253 | 106 | 189 | 194 | 90 |
| 2 | 210 | 214 | 97 | 180 | 181 | 92 |
| 5 | 163 | 167 | 100 | 160 | 157 | 93 |
| 10 | 140 | 140 | 95 | 132 | 127 | 92 |
| 15 | 119 | 116 | 91 | 115 | 113 | 89 |
| 20 | 103 | 103 | 87 | 99 | 97 | 86 |
| 30 | 88 | 87 | 85 | 86 | 85 | 77 |
| 45 | 81 | 81 | 84 | 76 | 76 | 74 |
| 60 | 77 | 77 | 76 | 74 | 73 | 81 |

TABLE 2
(1/10/11 Data)

| | CLOSED- | | VENTED- | | | | |
|---|---|---|---|---|---|---|---|
| | Empty | | Empty | | VENTED-Loaded | | |
| | Base | | Base | | Base | | |
| Time | of | Outside | of | Outside | of | Beads′ | Outside |
| (mins.) | Cup | Wall | Cup | Wall | Cup | Surface | Wall |
| −2 | 68 | 73 | 70 | 70 | 69 | 69 | 70 |
| 0 | 198 | 93 | 278 | 103 | 338 | 355 | 107 |
| 1 | 173 | 89 | 244 | 100 | 284 | 290 | 109 |
| 2 | 160 | 95 | 221 | 109 | 236 | 247 | 112 |
| 5 | 125 | 90 | 181 | 102 | 177 | 179 | 101 |
| 10 | 107 | 87 | 131 | 92 | 130 | 128 | 98 |
| 15 | 94 | 85 | 109 | 89 | 112 | 109 | 93 |
| 20 | 87 | 82 | 99 | 90 | 100 | 98 | 87 |
| 25 | | | 88 | 86 | 90 | 89 | 83 |
| 30 | | | 84 | 81 | 84 | 83 | 81 |

TABLE 3
(Jan. 16, 2011 Data)

| | VENTED-Empty | | VENTED-Loaded | | |
|---|---|---|---|---|---|
| Time | Cup | Outside | Base of | Beads' | Outside |
| (mins.) | Bottom | Wall | Cup | Surface | Wall |
| −2 | 68 | 67 | 70 | 69 | 69 |
| 0 | 307 | 88 | 305 | 327 | 96 |
| 1 | 252 | 92 | 281 | 301 | 99 |
| 2 | 219 | 91 | 248 | 245 | 106 |
| 3 | 195 | 96 | 229 | 228 | 105 |
| 4 | 176 | 85 | 205 | 205 | 109 |
| 5 | 167 | 87 | 193 | 188 | 100 |
| 6 | 153 | 80 | 176 | 167 | 91 |
| 7 | 144 | 79 | 165 | 151 | 91 |
| 8 | 135 | 85 | 152 | 145 | 89 |
| 9 | 126 | 86 | 142 | 135 | 86 |
| 10 | 121 | 84 | 134 | 132 | 87 |
| 11 | 117 | 81 | 130 | 122 | 82 |
| 12 | 110 | 82 | 123 | 117 | 80 |
| 13 | 106 | 82 | 121 | 117 | 76 |
| 14 | 102 | 78 | 116 | 114 | 85 |
| 15 | 99 | 79 | 112 | 106 | 85 |

Although the present invention has been described in detail with reference to certain embodiments, one skilled in the art will appreciate that the present invention can be practiced by other than the described embodiments, which have been presented for purposes of illustration and not of limitation. Therefore, the scope of the appended claims should not be limited to the description of the embodiments contained herein.

In certain embodiments, an assembly comprises: a receptacle including an inner vessel disposed within an outer vessel; and a plasticized elastomer disposed within the receptacle and formed from a polymeric carrier and a volatile compound. In certain embodiments, the polymeric carrier, of the assembly, is a polyvinyl ester, such as an ethylene vinylacetate copolymer. In certain embodiments, the volatile compound, of the assembly, is selected from the group consisting of: a fragrance; a medicament; an antimicrobial; and any combination thereof.

We claim:

1. A plasticized elastomer for dispensing a volatile compound comprising:
a polymeric carrier having a specific heat capacity above 1000 J/(Kg·° K); and
a volatile compound disposed in the polymeric carrier.

2. The plasticized elastomer of claim 1, wherein the polymeric carrier comprises a plurality of pendent ester moieties, wherein those pendent ester moieties rotate within said polymeric carrier when exposed to microwave energy.

3. The plasticized elastomer of claim 1, wherein the polymeric carrier has a melting point above 200 degrees Fahrenheit.

4. The plasticized elastomer of claim 1, wherein the polymeric carrier is Ethylene Vinyl Acetate.

5. The plasticized elastomer of claim 1, wherein the polymeric carrier is in a form of a paste, a gel, a spray, and a combination thereof.

6. The plasticized elastomer of claim 1, wherein the volatile compound is a selected from the group consisting of: a fragrance; a medicament; an antimicrobial; and any combination thereof.

7. The plasticized elastomer of claim 1, wherein the polymeric carrier is in pellet form.

8. An assembly for dispensing a volatile compound, the assembly including:
a receptacle;
a polymeric carrier disposed in the receptacle, wherein said polymeric carrier is stable at a temperature of about 400 degrees Fahrenheit; and
a volatile compound disposed in the polymeric carrier, wherein the volatile compound is released as a gas from when the polymeric carrier is heated to a temperature above room temperature.

9. The assembly of claim 8, wherein the polymeric carrier comprises a polyvinyl ester.

10. The assembly of claim 8, wherein the polymeric carrier comprises an ethylene vinylacetate copolymer.

11. The assembly of claim 8, wherein the volatile compound is selected from the group consisting of: a fragrance; a medicament; an antimicrobial; and any combination thereof.

12. The assembly of claim 8, wherein the receptacle is microwavable.

13. The assembly of claim 8, wherein:
the polymeric carrier is housed within a concavity of the receptacle; and
the concavity defines an open top to the receptacle.

14. The assembly of claim 8, wherein the receptacle has one or more vents disposed on a surface of the receptacle.

15. The assembly of claim 8, wherein:
the receptacle comprises an inner vessel disposed within an outer vessel;
the inner vessel is sealingly attached to a periphery of the outer vessel; and
the outer vessel is configured to allow microwave energy to pass through the outer vessel with a less than 20 degrees Fahrenheit temperature rise of a material of the outer vessel.

16. A method for dispensing a volatile compound, the method comprising:
exposing a plasticized elastomer to microwave energy to increase a temperature of the plasticized elastomer, wherein the plasticized elastomer includes a volatile compound disposed in a polymeric carrier;
and
allowing the plasticized elastomer to dissipate heat until a temperature of the plasticized elastomer reaches thermal equilibrium with an ambient temperature.

17. The method of claim 16, wherein the polymeric carrier is Ethylene Vinyl Acetate.

18. The method of claim 16, wherein the volatile compound is a selected from the group consisting of: a fragrance; a medicament; an antimicrobial; and any combination thereof.

19. The method of claim 16, wherein the plasticized elastomer is exposed to microwave energy for a period of time above 2 seconds.

20. The method of claim 16, further comprising disposing the plasticized elastomer in a microwavable receptacle prior to exposing the plasticized elastomer to the microwave energy.
